SEQUENCE LISTING (19 sequences)

SEQ ID NO: 1 - protein, 12 aa, Bacteriophage M13mp18
TDHTHNKGYANK

SEQ ID NO: 2 - protein, 12 aa, Bacteriophage M13mp18
TSHPSYYLTGSN

SEQ ID NO: 3 - protein, 12 aa, Bacteriophage M13mp18
SHQALQEMKLPM

SEQ ID NO: 4 - protein, 12 aa, Bacteriophage M13mp18
SMESLSKTHHYR

SEQ ID NO: 5 - protein, 12 aa, Bacteriophage M13mp18
KLHISKDHIYPT

SEQ ID NO: 6 - protein, 12 aa, Bacteriophage M13mp18
NRPDSAQFWLHH

SEQ ID NO: 7 - protein, 12 aa, Bacteriophage M13mp18
DPQNHNWTNKPA

SEQ ID NO: 8 - protein, 12 aa, Bacteriophage M13mp18
YLPHMLVHGSRH

SEQ ID NO: 9 - protein, 12 aa, Bacteriophage M13mp18
TYPVVGHQQNVM

SEQ ID NO: 10 - protein, 12 aa, Bacteriophage M13mp18
DIMPKLRDDVHN

SEQ ID NO: 11 - protein, 12 aa, Bacteriophage M13mp18
NAHTSNNVVAFP

SEQ ID NO: 12 - protein, 12 aa, Bacteriophage M13mp18
YGTSMTQSNWRH

SEQ ID NO: 13 - protein, 12 aa, Bacteriophage M13mp18
SYGSLQTRFGHI

SEQ ID NO: 14 - protein, 12 aa, Bacteriophage M13mp18
KFFNNTEATTRP

SEQ ID NO: 15 - protein, 12 aa, Bacteriophage M13mp18
NYALRDPVGQRY

SEQ ID NO: 16 - protein, 12 aa, Bacteriophage M13mp18
LPSVTEILGSNF

SEQ ID NO: 17 - protein, 12 aa, Bacteriophage M13mp18
TSAVTLTSDPTL

SEQ ID NO: 18 - protein, 12 aa, Bacteriophage M13mp18
QNFSQMMSIPRK

SEQ ID NO: 19 - protein, 12 aa, Bacteriophage M13mp18
AHQALQEMKLPM

(19) United States
(12) Patent Application Publication
(10) Pub. No.: US 2016/0177292 A1
(43) Pub. Date: Jun. 23, 2016

(54) METHOD FOR SCREENING CATALYTIC PEPTIDES USING PHAGE DISPLAY TECHNOLOGY

(71) Applicant: RESEARCH FOUNDATION OF THE CITY UNIVERSITY OF NEW YORK, New York, NY (US)

(72) Inventors: Hiroshi Matsui, New York, NY (US); Yoshiaki Maeda, New York, NY (US); Rein Ulijn, New York, NY (US)

(73) Assignee: Research Foundation of the City University of New York, New York, NY (US)

(21) Appl. No.: 14/902,622

(22) PCT Filed: Jul. 3, 2013
(86) PCT No.: PCT/US13/49218

§ 371 Date: Jan. 4, 2016

(51) Int. Cl. C12N 15/10

(52) U.S. Cl. CPC C12N 15/1037

(57) ABSTRACT

A method for screening catalytic peptides using phage display technology is disclosed. A compound is exposed to a phage library. If a peptide in the library catalyzes a reaction, a gel is formed about the phage that displays the peptide. The gel, including the first phage, is separated from un-reacted phages and released from the gel. The phage is then replicated and analyzed to determine the composition of the peptide that functioned as a catalyst.

### BACKGROUND OF THE INVENTION

The subject matter disclosed herein relates to methods for identifying peptides that are useful for catalyzing chemical reactions. Efficient and effective catalysis in various important chemical reactions requires high specificity to break or generate particular chemical bonds. In nature, enzymes are efficient catalysts, however they are complex and often unstable. It is desirable to develop catalytic peptides, which have much simpler molecular structures and are more stable, cost effective and more easily mass produced. However, catalytic peptides which can promote chemical bond generation/cleavage have been very rarely reported because of the lack of efficient methods to find or design them. An improved method of identifying such catalysts is therefore desired. The discussion above is merely provided for general background information and is not intended to be used as an aid in determining the scope of the claimed subject matter.

### BRIEF DESCRIPTION OF THE INVENTION

A method for screening catalytic peptides using phage display technology is disclosed. A compound is exposed to a phage library. If a peptide in the library catalyzes a reaction, a gel is formed about the phage that displays the peptide. The gel, including the first phage, is separated from un-reacted phages and released from the gel. The phage is then replicated and analyzed to determine the composition of the peptide that functioned as a catalyst. An advantage that may be realized in the practice of some disclosed embodiments of the method is that a wide range of peptides can be efficiently screened while no assumptions are made about the origins of catalysis.

A method for screening catalytic peptides using phage display technology is disclosed. A compound is exposed to a phage library. If a peptide in the library catalyzes a reaction, a gel is formed about the phage that displays the peptide. The gel, including the first phage, is separated from un-reacted phages and released from the gel. The phage is then replicated and analyzed to determine the composition of the peptide that functioned as a catalyst.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a flow diagram depicting an exemplary method for screening catalytic peptides using phage display technology;

FIG. 2 is schematic depiction of one example of the method of FIG. 1;

FIG. 3 is schematic depiction of another example of the method of FIG. 1; and

FIG. 4 depicts rate data of select peptides catalyzing a particular reaction that were identified using the method.

### DETAILED DESCRIPTION OF THE INVENTION

Efficient catalysis in water is a fundamental molecular process of all living systems that may be exploited in green chemistry, biotechnology and medicine. The de novo design and discovery of molecular catalysts for aqueous reactions (enzyme mimics) has been a longstanding challenge. Herein, we describe methodology that enables the selection of catalytic oligopeptides from sequence libraries based on their catalytic turnover. This is accomplished by catalytic gelation: by exposing vast peptide libraries, obtained through phage display, to precursors that catalytically convert to powerful gelators. When a phage display library is exposed to these precursors, phages that present catalytic sequences facilitate amide condensation and consequent localised gelation. The approach yields a number of peptides that are able to hydrolyze both ester and amide bonds showing modest rate enhancements. Unlike enzymes, these catalytic peptides do not rely on a rigid binding framework and are conformationally flexible. The isolated peptides can spontaneously access conformations that conceivably facilitate charge-relay between amino acids, similar to the catalytic mechanisms evolved by certain hydrolase enzymes but with minimal complexity. Their simplistic catalytic solution provides insights of relevance to the design of catalysts and may relate to the early precursors of enzymes. The disclosed method enables selection directly for catalysis amongst the random peptide sequences that are attached to phage particles. It should be emphasized that with this approach, there is no pre-determination about the need for good binding or the requirement for specific residues to be present, i.e., no assumptions are made about the origins of catalysis while creating a direct link between sequence and function.

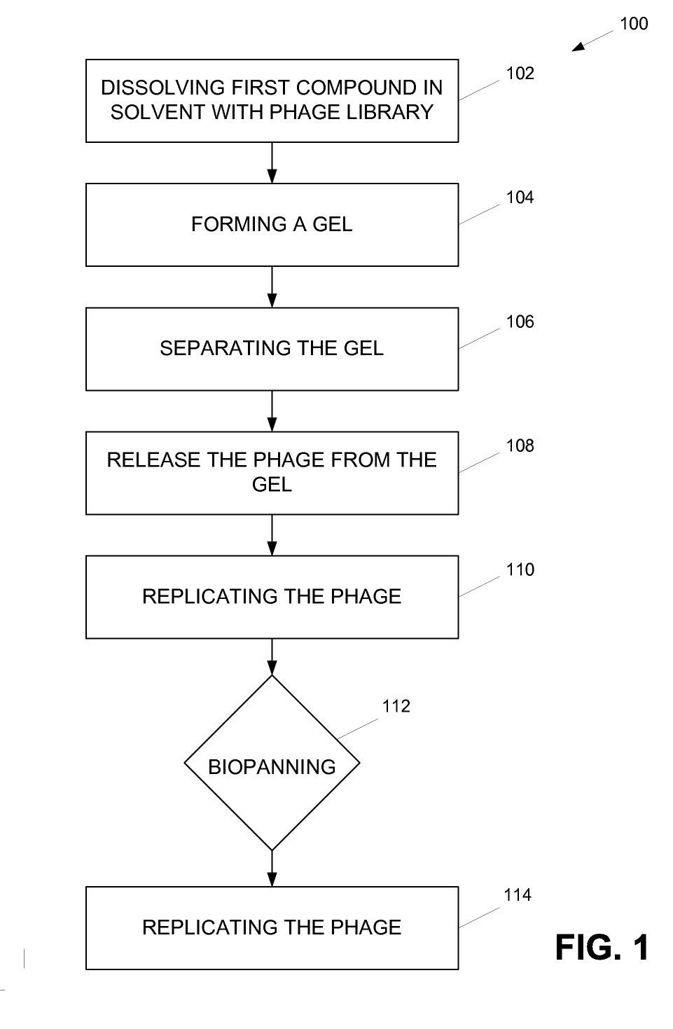

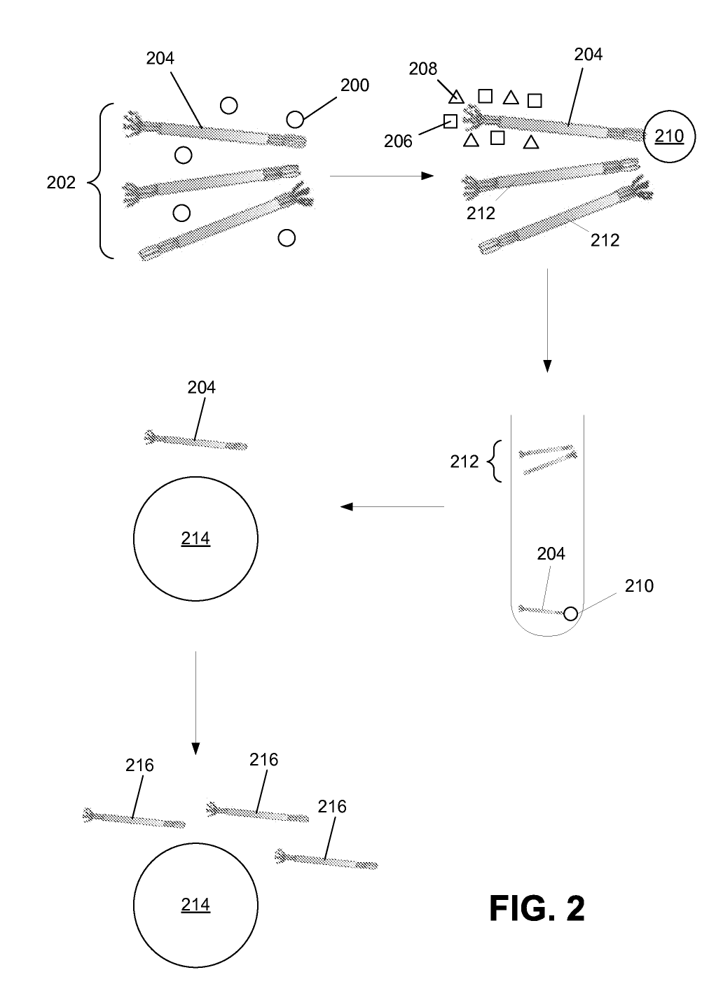

FIG. 1 is a flow diagram depicting an exemplary method 100 for screening catalytic peptides using phage display technology. FIG. 1 is described with reference to FIG. 2. The method 100 comprises a step 102 of dissolving a first compound 200 in a solvent that contains a phage library 202 that displays a plurality of peptides. The term “dissolving” includes both suspending and completely solvating. Phage libraries contain a vast (e.g. 10⁹) number of peptide sequences. The phage library 202 includes a first phage 204 with a first peptide. Phage libraries contain phages that display a variety of difference surface peptides—the composition of the surface peptides corresponds to the genetic sequence of the particular phage which displays that surface peptide.

In step 104 of method 100, a gel 210 is formed about the first phage 204 as a result of a reaction of the first compound 200, wherein the reaction is catalyzed by the first peptide of the first phage 204. The term gel refers to a self-assembled structure that results from the molecular self—assembly of the reaction products into nanoscale fibers, which in turn form a three-dimensional network that immobilizes water. In the exemplary embodiment of FIG. 2, the reaction is a degradation reaction that changes first compound 200 into a first product 206 and a second product 208. One or both of the first product 206 and/or the second product 208 are insoluble in the solvent. This insolubility causes the formation of the gel 210 in a region that is localized about the first phage 204. The un-reacted phages 212 present surface peptides that are different than the first peptide and do not catalyze the reaction. Accordingly, no gel is formed about the un-reacted phages 212.

In step 106 of method 100, the gel 210, including the first phage 204, is separated from un-reacted phages 212 of the phage library 202. A variety of separation techniques may be used including, for example, centrifugation or other separation techniques based on size and/or weight. In step 108 of method 100, the first phage 204 is released from the gel 210. In one embodiment, an enzyme (e.g. subtilisin) is used to effect the release. A variety of other gel-release agents are known in the art and are contemplated for use with the present invention.

In step 110 of method 100, the first phage 204 is replicated by exposing the first phage 204 to a bacterium 214 and permitting the formation of replicated first phages 216. Since the surface presentation of the first peptide is encoded in the genetic sequence of the first phage 204, the resulting replicated first phages 216 also present the first peptide.

In step 112, a biopanning decision is made. In step 112, a decision is made by comparing the current number of iterations of step 102-110 to the predetermined number. If the predetermined number has not been reached, the method 100 is re-executed beginning with step 102. If the predetermined number has been reached, then step 114 is executed. In step 114, the replicated first phages 216 are analyzed to determine the composition of the first peptide that catalyzed the reaction. In this fashion, a catalytic peptide has been identified.

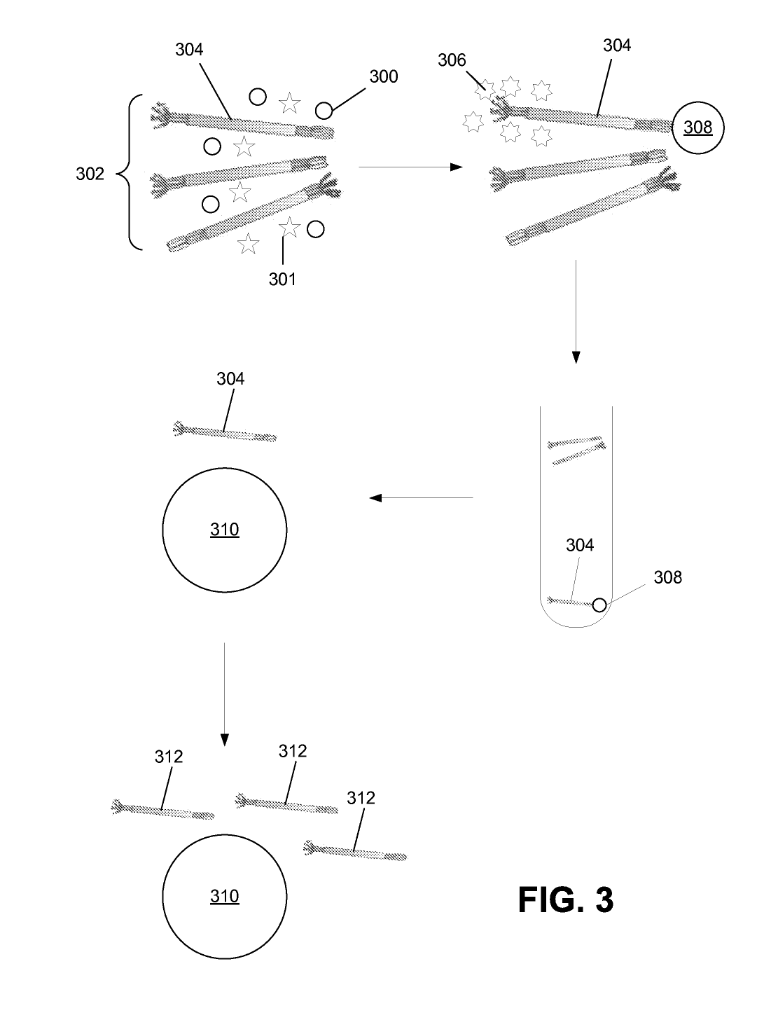

FIG. 3 depicts a similar embodiment, wherein the reaction is a synthesis reaction that changes a first compound 300 and a second compound 301 into a first product 306. Like the embodiment of FIG. 2, a phage library 302 is provided that includes a first phage 304. The first product 306 is insoluble in the solvent. The change in solubility may be caused by, for example, the relatively large molecular weight of the first product 306 relative to the relatively small molecular weights of the first compound 300 and second compound 301. This insolubility causes the formation of the gel 308 in a region that is localized about the first phage 304. In accordance with method 100, the gel 308 may then be separated, exposed to a bacterium 310 and replicated to form replicated first phages 312.

In one embodiment, the compounds (e.g. 200, 300, 301) comprise a carboxylic acid, an ester, a phosphate ester, an amine and/or an alcohol. In another embodiment, the compounds are amino acids or small peptides. For example, the first compound 200 may be a small peptide. In another embodiment, the first compound 300 may be a carboxylic acid (including an amino acid or peptide comprising amino acids) and the second compound 301 is an amine or alcohol. The resulting first product 306 is an amide or an ester, respectively. In other embodiments, the first compounds 200, 300 may be molecules other than amino acids or carboxylic acids.

### Example 1

Screening for Catalytic Function. The library of M13 phages, which displays approximately 2.7×10⁹ random peptide sequences, is incubated in the presence of the fully soluble gel precursors Fmoc-threonine (Fmoc-T) and leucine-methyl ester (L-OMe), previously shown to enable high yielding condensation to the Fmoc-TL-OMe gelator, driven by the free energy gain associated with self-assembly. Phages presenting peptide sequences that can catalyze amide condensation to form the gelator, would give rise to localized gel formation. Formation of localized gel surrounding the active peptide catalyst would then facilitate the separation and isolation of the catalytic phage by centrifugation.

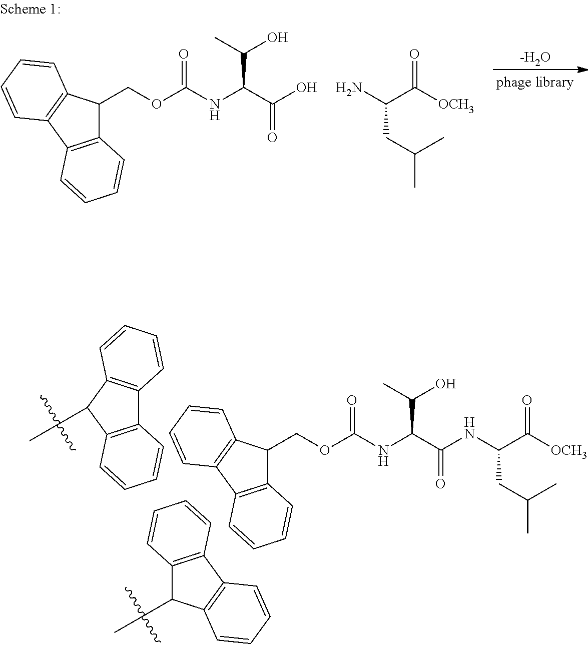

In order to remove the gel from the first phage to enable amplification, phages were subsequently incubated with subtilisin to hydrolyse the terminal methyl ester, and subsequently amplified to decode the relevant DNA sequence within the phage genome. This process revealed 18 peptides (Table 1).

TABLE 1

| | No. of | Name | |
|---|---|---|---|
| Sequence | Triads | in text | SEQ ID NO. |
| T D H T H N K G Y A N K | 8 | CP1 | SEQ ID NO. 1 |
| T S H P S Y Y L T G S N | 5 | CP2 | SEQ ID NO. 2 |
| S H Q A L Q E M K L P M | 2 | CP3 | SEQ ID NO. 3 |
| S M E S L S K T H H Y R | 16 | CP4 | SEQ ID NO. 4 |
| K L H I S K D H I Y P T | | | SEQ ID NO. 5 |
| N R P D S A Q F W L H H | | | SEQ ID NO. 6 |
| D P Q N H N W T N K P A | | | SEQ ID NO. 7 |
| Y L P H M L V H G S R H | | | SEQ ID NO. 8 |
| T Y P V V G H Q Q N V M | | | SEQ ID NO. 9 |
| D I M P K L R D D V H N | | | SEQ ID NO. 10 |
| N A H T S N N V V A F P | | | SEQ ID NO. 11 |
| Y G T S M T Q S N W R H | | | SEQ ID NO. 12 |
| S Y G S L Q T R F G H I | | | SEQ ID NO. 13 |
| K F F N N T E A T T R P | | | SEQ ID NO. 14 |
| N Y A L R D P V G Q R Y | | | SEQ ID NO. 15 |
| L P S V T E I L G S N F | | | SEQ ID NO. 16 |
| T S A V T L T S D P T L | | | SEQ ID NO. 17 |
| Q N F S Q M M S I P R K | | | SEQ ID NO. 18 |

Although there is no apparent sequence similarity between these ‘hits’, it is apparent that the majority of peptides that were selected contained amino acids that are typically associated with charge relay networks that enhance nucleophilicity, catalytic triads (a combination of a nucleophile, base and acid), one of biology's conserved approaches and is found in a range of amidases, esterases and lipases—such as serine proteases. Such triads consist of three precisely positioned and highly conserved residues: histidine (H), serine (S) and aspartic acid (D). Among the 18 peptides identified, 13 peptides contained at least one threonine/serine (T/S), and histidine (H). Of these 13 peptides, three peptides which also contained at least one glutamic/aspartic acid (E/D) were selected for further study in addition to a peptide lacking in E/D (CP2) (Table 2).

TABLE 2

| Name | Sequence | |
|---|---|---|
| CP1 | T D H T H N K G Y A N K | SEQ ID NO. 1 |
| CP2 | T S H P S Y Y L T G S N | SEQ ID NO. 2 |
| CP3 | S H Q A L Q E M K L P M | SEQ ID NO. 3 |
| CP4 | S M E S L S K T H H Y R | SEQ ID NO. 4 |
| CP3S1A | A H Q A L Q E M K L P M | SEQ ID NO. 19 |

Our method of catalytic gelation combined with phage display was successful in the identification of four different dodecapeptides, which catalyze the hydrolysis of ester and amide bonds under physiological conditions. Although they are conformationally flexible, these peptides can spontaneously access folds that agree with a catalytic mechanism of existing enzymes. The method is in stark contrast with conventional thought in that small peptides are successfully identified that lack the complicated and fragile three-dimensional structure through to be required for selectivity.

### Example 2

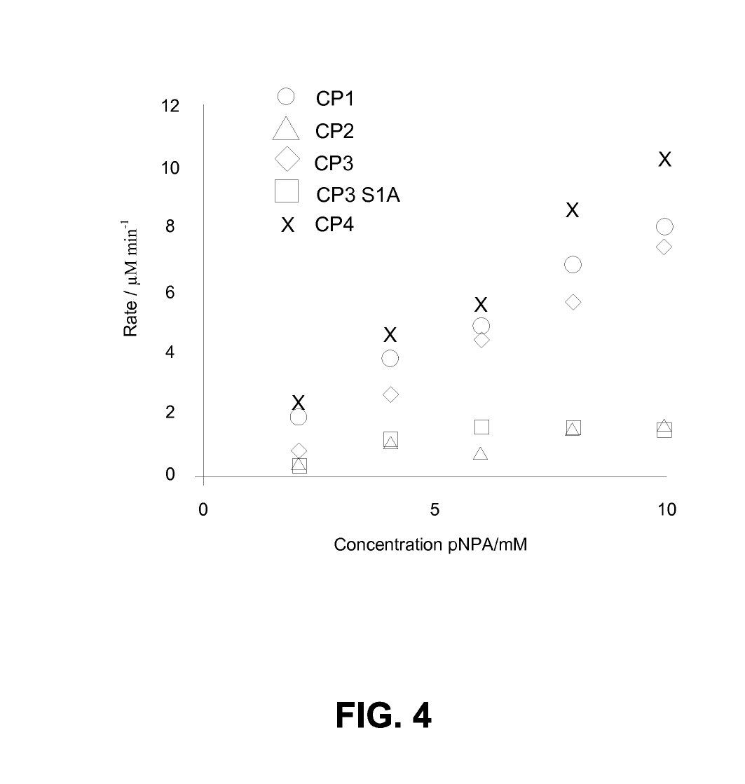

Catalytic Activity. To examine whether these dodeca peptides CP1-4 retained catalytic activity when free in solution (i.e. not attached to the phage filaments), they were produced by solid-phase peptide synthesis. In order to estimate the kinetic profiles of these catalysts, a readily hydrolysed ester was chosen (para-nitrophenyl acetate, pNPA), which is commonly used for comparative assessment of hydrolase activities. Although background hydrolytic activity is substantial, catalytic constants could be determined at varying substrate concentrations. These showed a linear profile within the concentration range studied (solubility of pNPA becomes limiting over 10 mM). FIG. 4 depicts rate data for the peptides of Table 2. The results suggest that the catalytic peptides cannot be described by Michaelis-Menten kinetics under these conditions. This is not surprising, as the high flexibility of the dodecapeptide and the absence of a well-defined binding pocket implies a minor role for substrate binding in catalysis. It seems reasonable that T or S residues play a role as nucleophiles in the catalysis. Indeed, when the S in CP3 was replaced by non-nucleophilic A (CP3S1A, SEQ ID NO. 19), the catalytic activity dropped dramatically. CP2, which lacks an acidic residue (although the terminal COOH group could contribute) showed the lowest catalytic activity.

Amide hydrolysis in water is an extremely challenging reaction with a free energy barrier giving rise to half lives in the range of 300 years. The free peptides were incubated with bovine serum albumin (BSA) (pH 8.0, room temperature). After incubation during 25 days at room temperature, cleaved protein fragments were indeed confirmed in sodium dodecyl sulfate polyacrylamide gel electrophoresis (SDS-PAGE) while in the absence of the peptides no observable digestion occurred. This result suggests that catalytic peptides identified here show a low level of amidolytic activity. Clearly, in the screening experiments amide condensation was significant during three days of incubation as it resulted in catalytic gelation (and condensation product could be detected).

### Example 3

Feasibility of a catalytic triad mechanism. It is conceivable that the CPs activity may be enhanced when peptides are attached to the phage where multiple peptides could conceivably contribute to the observed amide condensation. In hydrolases, the catalytic mechanism involves a charge-relay network between an alcohol bearing amino acid (S or T), histidine (H), and an acidic amino acid (D or E). For example, in the case of the serine protease subtilisin (PDB-ID: 1ST2), Ser-221 forms a hydrogen bond (3.21 Å) from the alcohol O(H) to the Nε of His-64, which is also connected via a hydrogen bond from the Nδ (H) to the (C)OO⁻ of Asp-32 (2.58 A and 3.37 Å, respectively).

Within the dodecapeptides CP1-4, there are often multiple possibilities for how the triad may be formed and the relative spatial arrangement of the amino acids is unclear from their primary sequence. In order to determine whether the peptides were able to temporarily fold into a conformation that allowed the catalytic triad to form, molecular dynamics (MD) simulations were carried out. These simulations identify which amino acids in the peptide are involved in forming the catalytic triad where multiple possibilities exist. The MD simulations reveal that the formation of a catalytic triad is possible in each case. For CP1, the triad is formed between D2, H3 and T4. A snapshots reveals that the key distances that define the triad are comparable to those observed in protease enzymes such as subtilisin and chymotrypsin (i.e., about 3 Å). While the peptides are clearly much more flexible than the relatively rigid active site of an enzyme—as evidenced by the variation in the key distances shown in the snapshot—the catalytic triad is able to be formed and the peptide does maintain this conformation for extended periods to support catalytic activity.

### Example 3

To confirm whether the discovered catalytic peptides, CP-1, CP-2, CP-3, and CP-4, can undergo the amidase activity to degrade proteins via amide bond cleavage in solution, these peptides are incubated with natural protein, bovine serum albumin (BSA). After 11 days at room temperature, cleaved protein fragments were indeed confirmed in the Coomassie brilliant blue (CBB)-stained sodium dodecyl sulfate polyacrylamide gel electrophoresis (SDS-PAGE). A control experiment in the absence of the catalytic peptides showed much weaker bands on SDS page, as a result of a low background level of protein degradation. Thus, it demonstrates that all catalytic peptides selected through the amide-gel biopanning plays a critical role in the amide bond-hydrolysis in the natural protein.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for screening catalytic peptides using phage display technology, the method comprising:
dissolving at least a first compound in a solvent that contains a phage library that displays a plurality of peptides, the phage library including a first phage with a first peptide;
forming a gel about at least the first phage as a result of a reaction of the first compound, wherein the reaction is catalyzed by the first peptide of the first phage;
separating the gel, including the first phage, from un-reacted phages of the phage library;
releasing the first phage from the gel;
replicating the first phage by exposing the first phage to a bacterium and permitting the formation of replicated first phages; and
analyzing the replicated first phages to determine a composition of the first peptide, thereby identifying the composition as a catalytic peptide that catalyzes the reaction of the first compound.

2. The method as recited in claim 1, wherein the step of separating the gel uses centrifugation.

3. The method as recited in claim 1, wherein the step of releasing the first phage from the gel uses an enzyme.

4. The method as recited in claim 1, wherein the solvent is water.

5. The method as recited in claim 1, wherein the bacterium is Escherichia coli.

6. The method as recited in claim 1, wherein the first compound comprises a carboxylic acid.

7. The method as recited in claim 1, wherein the first compound comprises at least one amino acid.

8. A method for screening catalytic peptides using phage display technology, the method comprising:
dissolving at least a first compound and a second compound in a solvent that contains a phage library that displays a plurality of peptides, the phage library including a first phage with a first peptide;
forming a gel about at least the first phage as a result of a reaction of the first compound with the second compound to form a first product, wherein the reaction is catalyzed by the first peptide of the first phage;
separating the gel, including the first phage, from un-reacted phages of the phage library;
releasing the first phage from the gel;
replicating the first phage by exposing the first phage to a bacterium and permitting the formation of replicated first phages; and
analyzing the replicated first phages to determine a composition of the first peptide, thereby identifying the composition as a catalytic peptide that catalyzes the reaction of the first compound with the second compound.

9. The method as recited in claim 8, wherein the first compound comprises at least one amino acid.

10. The method as recited in claim 9, wherein the second compound comprises at least one amino acid.

11. The method as recited in claim 8, wherein the first compound comprises a carboxylic acid.

12. The method as recited in claim 8, wherein the first compound comprises a carboxylic acid, the second compound comprises an amine and the first product is an amide.

13. The method as recited in claim 8, wherein the first compound comprises a carboxylic acid, the second compound comprises an alcohol and the first product is an ester.

14. A method for screening catalytic peptides using phage display technology, the method comprising:
dissolving at least a first compound in a solvent that contains a phage library that displays a plurality of peptides, the phage library including a first phage with a first peptide;
forming a gel about at least the first phage as a result of a reaction of the first compound, wherein the reaction is catalyzed by the first peptide of the first phage;
separating the gel, including the first phage, from un-reacted phages of the phage library;
releasing the first phage from the gel;
replicating the first phage by exposing the first phage to a bacterium and permitting the formation of replicated first phages;
biopanning the replicated first phages at least once; and
analyzing the replicated first phages to determine a composition of the first peptide, thereby identifying the composition as a catalytic peptide that catalyzes the reaction of the first compound.

15. The method as recited in claim 14, wherein the first compound comprises a carboxylic acid, the second compound comprises an amine and the first product is an amide.

16. The method as recited in claim 14, wherein the first compound comprises a carboxylic acid, the second compound comprises an alcohol and the first product is an ester.

17. The method as recited in claim 14, wherein the first compound comprises an amino acid, the second compound comprises an amino acid and the first product is a peptide linked by a newly formed amide between the first compound and the second compound.
